(19) United States
(12) Patent Application Publication
(10) Pub. No.: US 2006/0217777 A1
(43) Pub. Date: Sep. 28, 2006

(54) Apparatus and methods of monitoring cardiac activity utilizing implantable shroud-based electrodes

(76) Inventors: James Strom, Arden Hills, MN (US); Scott J. Brabec, Elk River, MN (US); John E. Nicholson, Blaine, MN (US); Michael D. Fletcher, Minneapolis, MN (US); Thomas H. Adamski, Andover, MN (US); Christopher C. Stancer, Prescott, WI (US)

Correspondence Address: MEDTRONIC, INC., 710 MEDTRONIC PARK, MINNEAPOLIS, MN, 55432-9924, US

(21) Appl. No.: 11/085,843

(22) Filed: Mar. 22, 2005

(51) Int. Cl. A61N 1/375

(52) U.S. Cl. 607/36

(57) ABSTRACT

The present invention provides a subcutaneous (or submuscular) single or multiple-electrode array that provides various embodiments of a compliant surround shroud coupled to a peripheral portion of an implantable medical device (IMD). The shroud incorporates a plurality of substantially planar electrodes mechanically coupled within recessed portions of the shroud. These electrodes electrically couple to IMD circuitry to monitor cardiac activity of a subject. Temporal recordings of the detected cardiac activity are referred to herein as an extra-cardiac electrogram (EC-EGM). The recordings can be stored upon computer readable media within an IMD at various resolution (e.g., continuous beat-by-beat, periodic, triggered, mean value, average value, etc.). Real time or stored EC-EGM signals can be provided to remote equipment via telemetry. For example, when telemetry, or programming, head of an IMD programming apparatus is positioned within range of an IMD the programmer receives some or all of the EC-EGM signals.

### CROSS REFERENCE TO RELATED APPLICATION

The present patent document is related to co-pending non-provisional patent application Ser. No. ______, entitled, “METHODS OF FABRICATION OF SHROUD-BASED ELECTRODES FOR MONITORING CARDIAC ACTIVITY,” filed on even date herewith and the contents of which are hereby incorporated by reference.

### FIELD OF THE INVENTION

The present invention relates generally to implantable medical devices and more particularly to a subcutaneous multiple electrode sensing and recording system for acquiring electrocardiographic data and waveform tracings from an implanted medical device without the need for or use of surface (skin) electrodes. More particularly, the present invention relates to implantable devices that are equipped with a shroud member adapted to receive at least one electrode that is operatively coupled to circuitry to monitor cardiac activity.

### BACKGROUND OF THE INVENTION

The electrocardiogram (ECG) is commonly used in medicine to determine the status of the electrical conduction system of the human heart. As practiced the ECG recording device is commonly attached to the patient via ECG leads connected to pads arrayed on the patient's body so as to achieve a recording that displays the cardiac waveforms in any one of 12 possible vectors.

Since the implantation of the first cardiac pacemaker, implantable medical device technology has advanced with the development of sophisticated, programmable cardiac pacemakers, pacemaker-cardioverter-defibrillator arrhythmia control devices and drug administration devices designed to detect arrhythmias and apply appropriate therapies. The detection and discrimination between various arrhythmic episodes in order to trigger the delivery of an appropriate therapy is of considerable interest. Prescription for implantation and programming of the implanted device are based on the analysis of the PQRST electrocardiogram (ECG) that currently requires externally attached electrodes and the electrogram (EGM) that requires implanted pacing leads. The waveforms are usually separated for such analysis into the P-wave and R-wave in systems that are designed to detect the depolarization of the atrium and ventricle respectively. Such systems employ detection of the occurrence of the P-wave and R-wave, analysis of the rate, regularity, and onset of variations in the rate of recurrence of the P-wave and R-wave, the morphology of the P-wave and R-wave and the direction of propagation of the depolarization represented by the P-wave and R-wave in the heart. The detection, analysis and storage of such EGM data within implanted medical devices are well known in the art. For example, S-T segment changes can be used to detect an ischemic episode. Acquisition and use of ECG tracing(s), on the other hand, has generally been limited to the use of an external ECG recording machine attached to the patient via surface electrodes of one sort or another.

The aforementioned ECG systems that utilize detection and analysis of the PQRST complex are all dependent upon the spatial orientation and number of electrodes available in or around the heart to pick up the depolarization wave front

As the functional sophistication and complexity of implantable medical device systems increased over the years, it has become increasingly more important for such systems to include a system for facilitating communication between one implanted device and another implanted device and/or an external device, for example, a programming console, monitoring system, or the like. For diagnostic purposes, it is desirable that the implanted device be able to communicate information regarding the device's operational status and the patient's condition to the physician or clinician. State of the art implantable devices are available which can even transmit a digitized electrical signal to display electrical cardiac activity (e.g., an ECG, EGM, or the like) for storage and/or analysis by an external device. The surface ECG, in fact, has remained the standard diagnostic tool since the very beginning of pacing and remains so today.

To diagnose and measure cardiac events, the cardiologist has several tools from which to choose. Such tools include twelve-lead electrocardiograms, exercise stress electrocardiograms, Holter monitoring, radioisotope imaging, coronary angiography, myocardial biopsy, and blood serum enzyme tests. Of these, the twelve-lead electrocardiogram (ECG) is generally the first procedure used to determine cardiac status prior to implanting a pacing system; thereafter, the physician will normally use an ECG available through the programmer to check the pacemaker's efficacy after implantation. Such ECG tracings are placed into the patient's records and used for comparison to more recent tracings. It must be noted, however, that whenever an ECG recording is required (whether through a direct connection to an ECG recording device or to a pacemaker programmer), external electrodes and leads must be used.

Unfortunately, surface electrodes have some serious drawbacks. For example, electrocardiogram analysis performed using existing external or body surface ECG systems can be limited by mechanical problems and poor signal quality. Electrodes attached externally to the body are a major source of signal quality problems and analysis errors because of susceptibility to interference such as muscle noise, power line interference, high frequency communication equipment interference, and baseline shift from respiration or motion. Signal degradation also occurs due to contact problems, ECG waveform artifacts, and patient discomfort. Externally attached electrodes are subject to motion artifacts from positional changes and the relative displacement between the skin and the electrodes. Furthermore, external electrodes require special skin preparation to ensure adequate electrical contact. Such preparation, along with positioning the electrode and attachment of the ECG lead to the electrode needlessly prolongs the pacemaker follow-up session. One possible approach is to equip the implanted pacemaker with the ability to detect cardiac signals and transform them into a tracing that is the same as or comparable to tracings obtainable via ECG leads attached to surface electrodes.

Previous art describes how to monitor electrical activity of the human heart for diagnostic and related medical purposes. U.S. Pat. No. 4,023,565 issued to Ohlsson describes circuitry for recording ECG signals from multiple lead inputs. Similarly, U.S. Pat. No. 4,263,919 issued to Levin, U.S. Pat. No. 4,170,227 issued to Feldman, et al, and U.S. Pat. No. 4,593,702 issued to Kepski, et al, describe multiple electrode systems, which combine surface EKG signals for artifact rejection.

The primary use for multiple electrode systems in the prior art is vector cardiography from ECG signals taken from multiple chest and limb electrodes. This is a technique whereby the direction of depolarization of the heart is monitored, as well as the amplitude. U.S. Pat. No. 4,121,576 issued to Greensite discusses such a system.

Numerous body surface ECG monitoring electrode systems have been employed in the past in detecting the ECG and conducting vector cardiographic studies. For example, U.S. Pat. No. 4,082,086 to Page, et al., discloses a four electrode orthogonal array that may be applied to the patient's skin both for convenience and to ensure the precise orientation of one electrode to the other. U.S. Pat. No. 3,983,867 to Case describes a vector cardiography system employing ECG electrodes disposed on the patient in normal locations and a hex axial reference system orthogonal display for displaying ECG signals of voltage versus time generated across sampled bipolar electrode pairs.

U.S. Pat. No. 4,310,000 to Lindemans, U.S. Pat. No. 6,512,940 to Brabec et al., U.S. Pat. No. 6,522,915 to Ceballos et al., U.S. Pat. Nos. 4,729,376 and 4,674,508 to DeCote, incorporated herein by reference, disclose related art the contents of each which are hereby incorporated by reference herein.

Moreover, in regard to subcutaneously implanted EGM electrodes, the aforementioned Lindemans U.S. Pat. No. 4,310,000 discloses one or more reference sensing electrode positioned on the surface of the pacemaker case as described above. U.S. Pat. No. 4,313,443 issued to Lund describes a subcutaneously implanted electrode or electrodes for use in monitoring the ECG.

Finally, U.S. Pat. No. 5,331,966 to Bennett, incorporated herein by reference, discloses a method and apparatus for providing an enhanced capability of detecting and gathering electrical cardiac signals via an array of relatively closely spaced subcutaneous electrodes (located on the body of an implanted device).

### SUMMARY OF THE INVENTION

The present invention provides a leadless subcutaneous (or submuscular) single or multiple-electrode array that provides various embodiments of a compliant surround shroud coupled to a peripheral portion of an implantable medical device (IMD). The shroud incorporates a plurality of substantially planar electrodes mechanically coupled within recessed portions of the shroud. These electrodes electrically couple to circuitry of an IMD and are adapted to detect cardiac activity of a subject. Temporal recordings of the detected cardiac activity are referred to herein as an extra-cardiac electrogram (EC-EGM). The recordings can be stored upon computer readable media within an IMD at various resolution (e.g., continuous beat-by-beat, periodic, triggered, mean value, average value, etc.). Real time or stored EC-EGM signals can be provided to remote equipment via telemetry. For example, when telemetry, or programming, head of an IMD programming apparatus is positioned within range of an IMD the programmer receives some or all of the EC-EGM signals.

The present invention provides improved apparatus and methods for reliably collecting EC-EGM signals for use or collection in conjunction with diverse IMDs (e.g., implantable pacemakers having endocardial leads, implantable cardioverter-defibrillators or ICDs, drug delivery pumps, subcutaneous ICDs, submuscular ICDs, brain stimulation devices, nerve stimulation devices, muscle stimulation devices and the like).

The invention can be implemented employing suitable sensing amplifiers, switching circuits, signal processors, and memory to process the EC-EGM signals collected between any selected pair or pairs of the electrodes deployed in an array around the periphery of an IMD to provide a leadless, orientation-insensitive means for receiving the EC-EGM signals from the heart.

The shroud can comprise a non-conductive, bio-compatible material such as any appropriate resin-based material, urethane polymer, silicone, or relatively soft urethane that retains its mechanical integrity during manufacturing and prolonged exposure to body fluids. The shroud placed around the peripheral portions of an IMD can utilize a number of configurations (e.g., two, three, four recesses) for individual electrodes. However, a four-electrode embodiment appears to provide an improved signal-to-noise ratio than the three-electrode embodiment. And, embodiments having a single electrode pair appear much more sensitive to appropriate orientation of the device relative to the heart than embodiments having more than a single pair of electrodes. Of course, embodiments of the invention using more than four electrodes increase complexity without providing a significant improvement in signal quality.

Embodiments having electrodes connected to three sense-amplifiers that are hardwired to three electrodes can record simultaneous EC-EGM signals. Alternative embodiments employ electrodes on the face of the lead connector, or header module, and/or major planar face(s) of the pacemaker that may be selectively or sequentially coupled in one or more pairs to the terminals of one or more sense amplifiers to pick up, amplify and process the EC-EGM signals across each electrode pair. In one aspect, the EC-EGM signals from a first electrode pair are stored and compared to other electrode pair(s) in order to determine the optimal sensing vector. Following such an optimization procedure, the system can be programmed to chronically employ the selected subcutaneous EC-EGM signal vector.

The three electrode and three amplifier embodiment offers several advantages including ability to sense cardiac activity in virtually every direction by adjusting the selected sensing vector.

Prior art patent U.S. Pat. No. 5,331,966 had electrodes placed on the face of the implanted pacemaker. When facing muscle tissue, the electrodes were apt to detect myopotentials and were susceptible to baseline drift. The present invention minimizes myopotentials and allows the device to be implanted in a variety of subcutaneous or submuscular locations of a patient's thorax by providing maximum electrode separation and minimal signal variation due to various orientation of an IMD within a surgically-created pocket because the electrodes are placed on the surround shroud in such a way as to maximize the distance between electrode pairs. The shroud provides insulation from the typically metallic IMD casing due to the insulative properties of the compliant shroud and recesses where the electrodes are mechanically coupled. The electrode placement maintains a maximum and equal distance between the electrode pairs. Such spacing with the four-electrode embodiment maintains maximum average signal due to the fact that the spacing of the two vectors is equal and the angle between these vectors is 90°, as known in the art and as predicted via mathematical modeling. Such orthogonal spacing of the electrode pairs also minimizes signal variation. An alternate three-electrode embodiment provides the electrodes arranged within the surround shroud in an equilateral triangle along the perimeter of the implanted pacemaker. Vectors in this embodiment can be combined to provide adequate sensing of cardiac signals.

Certain embodiments of the invention utilize substantially planar electrodes having a protective coating on at least the exposed surfaces, or all surfaces of the planar portions and the elongated conductor portion thereof. In the event that an increase in surface area of the electrodes is desired, a layer of material can be used (e.g., titanium nitride, platinum black, or the like).

In one aspect of these embodiments the substantially planar electrodes include mechanical features designed to cooperatively interlock with opposing features of the shroud. One type of such a mechanical feature includes one or more apertures formed in a portion of the planar electrode and an opposing, preferably similarly shaped protrusion or boss member disposed in an electrode-receiving recess. While not required to practice the present invention, the protrusion or boss member can be formed of a thermoplastic resin material that is susceptible of ultrasonic bonding. This type of bonding process is well known in the art and simply requires an ultrasonic head (or horn) be applied briefly to effective melt a portion of the boss member and thus produce an enlarged head portion that overlies and mechanically couples a portion of the planar electrode to the recess. Of course, in lieu of or in addition to the ultrasonic bonding just described the boss member can include a passive fixation feature such as a pre-split, separated head portion that contracts through the aperture and expands once it passes through the aperture.

In another form of the foregoing aspect of the invention a peripheral portion of a planar electrode is configured to engage a peripheral portion of the recess. In one embodiment of this form of the invention an enlarged portion of the elongated conductor used to couple the planar electrode to circuitry disposed within an IMD engages a wiring aperture thus providing mechanical interlocking engagement with a portion of the planar electrode and a pathway to the interior portion of the shroud (i.e., adjacent the periphery of the IMD housing).

With respect to the shroud member, besides the interlocking feature just described, in one embodiment the interior portion of the shroud includes a pre-configured conductor-receiving pathway from an area adjacent a recess to an area adjacent a conductor termination location. Optionally, the pre-configured conductor pathway can include friction-type mechanical engagement features (e.g., fingers, plates, clips, etc.) to ensure compact and accurate assembly during initial fabrication processing.

With respect to the elongated conductor coupling the planar electrodes to operative circuitry within an IMD, the assembly can comprise a unitary member stamped from a plate of conductive material such as titanium. In one embodiment the unitary member comprises a pre-shaped partially serpentine workpiece having a slightly curvilinear (i.e., substantially planar) major plate portion, a transition portion, and a partially serpentine portion adapted to cooperate with the configuration of the pre-configured conductor pathway.

For mass production of assemblies according to the invention a unique electrode piecepart can be fabricated for each unique conductor pathway and recess shape and configuration (including any of the variety of diverse mechanical interlocking features described hereinabove). Besides manufacturing processes such as metal stamping, the metallic electrode member(s) can be fabricating using electron discharge machining (EDM), laser cutting, or the like. It is desirable that the electrode assemblies are pre-configured (at least in a two-dimensional (2D) manner) so that little or no mechanical deformation or bending is required to fit each assembly into a shroud member. In addition, due to pre-configuring the parts the bends occur in a highly predictable manner and retain relatively little, if any, energy due to the spring-constant of the metal used to form the parts. In the event that electrical insulation or a dielectric layer becomes necessary or desirable, the major elongated portion of an electrode assembly can be coated with a material such as paralyne or similar.

In addition to permanent surface coating for the electrodes such as titanium nitride for titanium electrode assemblies, the surfaces of the electrodes may require temporary protection during manual handling to prevent contamination. A coating, such as may be provided by Dexamethazone Sodium Phosphate, NaCL (salts) and sugar solutions, provides such protection as well as enhancing the wetting of the electrode surface after implant. Conductive hydro gels, applied wet and allowed to dry, may also be applied to the electrode surfaces to protect them from damage during handling while helping to prevent contamination.

Electrode assemblies according to the invention can be used for chronic or acute EC-EGM signal sensing collection and attendant heart rate monitoring, capture detection, arrhythmia detection, and the like as well as detection of myriad other cardiac insults (e.g., ischemia monitoring using S-T segment changes, pulmonary edema monitoring based upon impedance changes).

Electrode assemblies according to the invention increase ease of fabrication due to the pre-formed parts and mechanical interlocking features and increase signal-to-noise ratio due to the relatively large surface area of the planar electrodes. In addition, manufacturing yield improvements are realized due to enhanced alignment of the proximal end portions of the pre-formed elongated conductors relative to multi-polar electrical feedthrough arrays. Yield improvements due to the unique length and shape of each discrete electrode part are also realized when practicing the invention. That is, a person assembling an IMD or during a pre-assembly inspection, according to certain aspects of the invention, can expect the feedthrough terminations and the terminations to be accurately inserted and aligned per a desired specification. The invention also offers advantages for automating all or a part of the fabrication process including laser welding the terminations together.

The surface of the electrode can be treated with one or more electrode coatings to enhance signal-conducting, de- and re-polarization sensing properties, and to reduce polarization voltages (e.g., platinum black, titanium nitride, titanium oxide, iridium oxide, platinum black, carbon, etc.). That is the surface area of the electrode surfaces may be increased by techniques known in the art. For example, the surfaces may be roughened or texturized or otherwise made porous and/or microporous and/or can be coated with such materials as just described and equivalents thereof. All of these materials are known to increase the true electrical surface area to improve the efficiency of electrical performance by reducing wasteful electrode polarization, among other advantages. The materials can be applied using any of a variety of techniques such as by sputtering, electron beam deposition, CVD or the like. These coatings can become more important over time as such enhancing coatings can help as the electrodes (typically) become encapsulated in scar tissue and thus at least indirectly contact with the body tissue. Such indirect tissue contact can damp the cardiac signals thus negatively affecting the sensing and detection ability of uncoated electrode(s).

Many of the embodiments of the inventive electrodes herein can provide a continuous electrical path free of welds or bonds on a portion of the planar electrode, the transition portion, the elongated conductor or the distal tip portion. Moreover, the electrode assembly according to the invention anchors to a shroud member free of any chemical or adhesive bonding materials that can cause excursions due to electro-active specie release to the electrode surface or portions thereof.

These and other advantageous aspects of the invention will be appreciated by those of skill in the art after studying the invention herein described, depicted and claimed. In addition, persons of skill in the art will appreciate insubstantial modifications of the invention that are intended to be expressly covered by the appended claims.

### BRIEF DESCRIPTION OF THE DRAWINGS

FIG. 1 schematically illustrates a body-implantable device system in accordance with one embodiment of the invention, including a hermetically sealed device implanted in a patient wirelessly coupled to an external programming unit.

FIG. 2 depicts a perspective view of an external programming unit.

FIG. 3 depicts a simplified block diagram of an exemplary IMD.

FIG. 4 depicts an exploded view of internal circuitry of an exemplary IMD, which can readily adapt to the apparatus and sensing methods of the present invention.

FIG. 5 depicts a perspective view with certain parts removed for ease of reference of a shroud of one embodiment of the invention.

FIG. 6 depicts another perspective view similar to the view of FIG. 5 except from a slightly different angle and illustrating enlarged components of a shroud of one embodiment of the invention.

FIG. 7 is an exploded view depicting an exemplary electrode adjacent an electrode receiving recess according to one embodiment of the invention.

FIG. 8 depicts in perspective view a cross-sectional portion of an electrode-receiving recess having an electrode coupled therein according to one embodiment of the invention.

FIG. 9 is an elevational view in cross-section of the electrode-receiving recess having an electrode coupled therein according to one embodiment of the invention.

FIG. 10 is an elevational view of the juxtaposition of a plurality of distal end portions of the elongated conductors of the electrodes following assembly of a shroud assembly according to one embodiment of the invention.

FIG. 11 illustrates just four configurations for the spacing and orientation of electrode placement around the periphery of an IMD of a given shape and size.

### DETAILED DESCRIPTION OF THE DRAWINGS

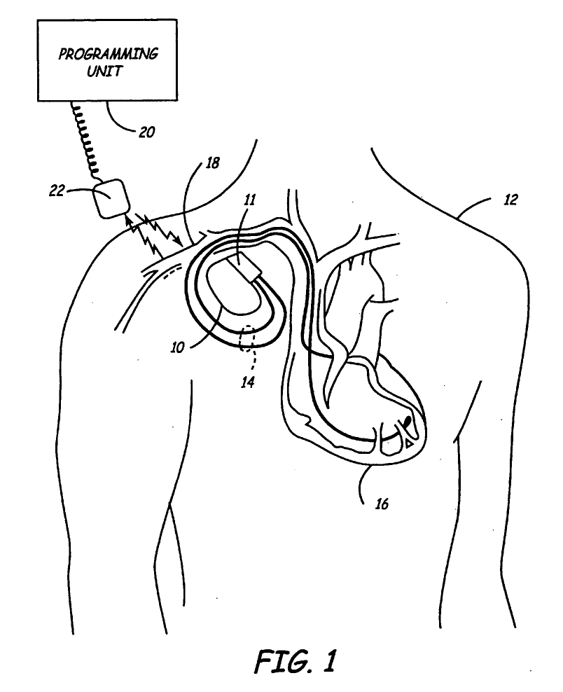

FIG. 1 is an illustration of an implantable medical device system adapted for use in accordance with the present invention. The medical device system shown in FIG. 1 includes an implantable device 10—a pacemaker in this embodiment—which has been implanted in a patient 12. In accordance with conventional practice in the art, pacemaker 10 is housed within a hermetically sealed, biologically inert outer casing, which may itself be conductive so as to serve as an indifferent electrode in the pacemaker's pacing/sensing circuit. One or more pacemaker leads, collectively identified with reference numeral 14 in FIG. 1 are electrically coupled to pacemaker 10 in a conventional manner and extend into the patient's heart 16 via a vein 18. Disposed generally near the distal end of leads 14 are one or more exposed conductive electrodes for receiving electrical cardiac signals and/or for delivering electrical pacing stimuli to heart 16. As will be appreciated by those of ordinary skill in the art, leads 14 may be implanted with its distal end situated in the atrium and/or ventricle of heart 16.

Although the present invention will be described herein in one embodiment which includes a pacemaker, those of ordinary skill in the art having the benefit of the present disclosure will appreciate that the present invention may be advantageously practiced in connection with numerous other types of implantable medical device systems, and indeed in any application in which it is desirable to provide a communication link between two physically separated components, such as may occur during transtelephonic monitoring.

Also depicted in FIG. 1 is an external programming unit 20 for non-invasive communication with implanted device 10 via uplink and downlink communication channels, to be hereinafter described in further detail. Associated with programming unit 20 is a programming head 22, in accordance with conventional medical device programming systems, for facilitating two-way communication between implanted device 10 and programmer 20. In many known implantable device systems, a programming head such as that depicted in FIG. 1 is positioned on the patient's body over the implant site of the device (usually within 2- to 3-inches of skin contact), such that one or more antennae within the head can send RF signals to, and receive RF signals from, an antenna disposed within the hermetic enclosure of the implanted device or disposed within the connector block of the device, in accordance with common practice in the art.

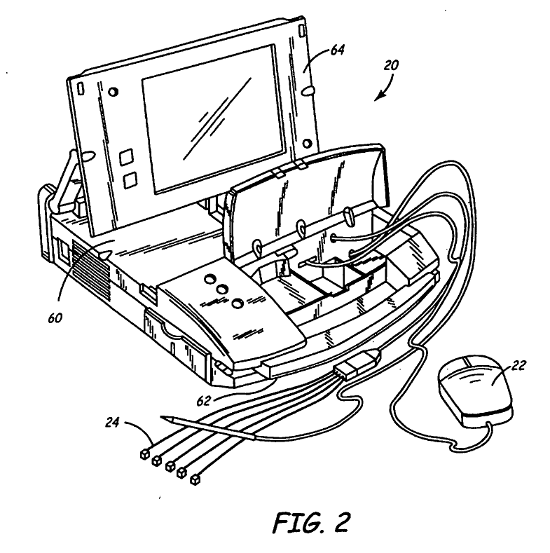

In FIG. 2, there is shown a perspective view of programming unit 20 in accordance with the presently disclosed invention. Internally, programmer 20 includes a processing unit (not shown in the Figures) that in accordance with the presently disclosed invention is a personal computer type motherboard, e.g., a computer motherboard including an Intel family of microprocessor(s) and related circuitry such as digital memory. The details of design and operation of the programmer's computer system will not be set forth in detail in the present disclosure, as it is believed that such details are well-known to those of ordinary skill in the art.

Referring to FIG. 2, programmer 20 comprises an outer housing 60, which is preferably made of thermal plastic or another suitably rugged yet relatively light-weight material. A carrying handle, designated generally as 62 in FIG. 2, is integrally formed into the front of housing 60. With handle 62, programmer 20 can be carried like a briefcase.

An articulating display screen 64 is disposed on the upper surface of housing 60. Display screen 64 folds down into a closed position (not shown) when programmer 20 is not in use, thereby reducing the size of programmer 20 and protecting the display surface of display 64 during transportation and storage thereof.

A floppy disk drive is disposed within housing 60 and is accessible via a disk insertion slot (not shown). A hard disk drive is also disposed within housing 60, and it is contemplated that a hard disk drive activity indicator, (e.g., an LED, not shown) could be provided to give a visible indication of hard disk activation.

Those with ordinary skill in the art know it is often desirable to provide a means for determining the status of the patient's conduction system. Normally, programmer 20 is equipped with external ECG leads 24. It is these leads, which are rendered redundant by the present invention.

In accordance with the present invention, programmer 20 is equipped with an internal printer (not shown) so that a hard-copy of a patient's ECG or of graphics displayed on the programmer's display screen 64 can be generated. Several types of printers, such as the AR-100 printer available from General Scanning Co., are known and commercially available.

In the perspective view of FIG. 2, programmer 20 is shown with articulating display screen 64 having been lifted up into one of a plurality of possible open positions such that the display area thereof is visible to a user situated in front of programmer 20. Articulating display screen is preferably of the LCD or electro-luminescent type, characterized by being relatively thin as compared, for example, a cathode ray tube (CRT) or the like.

Display screen 64 is operatively coupled to the computer circuitry disposed within housing 60 and is adapted to provide a visual display of graphics and/or data under control of the internal computer.

Programmer 20 described herein with reference to FIG. 2 is described in more detail in U.S. Pat. No. 5,345,362 issued to Thomas J. Winkler, entitled “Portable Computer Apparatus With Articulating Display Panel,” which patent is hereby incorporated herein by reference in its entirety. The Medtronic Model 9790 programmer is the implantable device-programming unit with which the present invention may be advantageously practiced. More recently the Medtronic Model 2090 programmer has been commercialized and other, longer-range programming devices are now becoming available. In this regard U.S. Pat. No. 6,169,925 is hereby incorporated by reference herein as an example of longer range, headless telemetry system for IMDs.

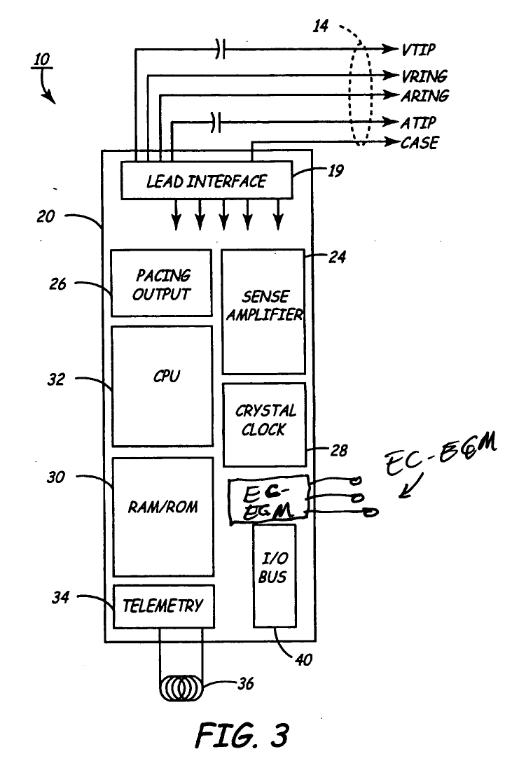

FIG. 3 is a block diagram of the electronic circuitry that makes up pulse generator 10 in accordance with the presently disclosed invention. As can be seen from FIG. 3, pacemaker 10 comprises a primary stimulation control circuit 20 for controlling the device's pacing and sensing functions. The circuitry associated with stimulation control circuit 20 may be of conventional design, in accordance, for example, with what is disclosed U.S. Pat. No. 5,052,388 issued to Sivula et al., “Method and apparatus for implementing activity sensing in a pulse generator.” To the extent that certain components of pulse generator 10 are conventional in their design and operation, such components will not be described herein in detail, as it is believed that design and implementation of such components would be a matter of routine to those of ordinary skill in the art. For example, stimulation control circuit 20 in FIG. 3 includes sense amplifier circuitry 24, stimulating pulse output circuitry 26, a crystal clock 28, a random-access memory and read-only memory (RAM/ROM) unit 30, and a central processing unit (CPU) 32, all of which are well-known in the art.

Pacemaker 10 also includes internal communication circuit 34 so that it is capable communicating with external programmer/control unit 20, as described in FIG. 2 in greater detail.

With continued reference to FIG. 3, pulse generator 10 is coupled to one or more leads 14 which, when implanted, extend transvenously between the implant site of pulse generator 10 and the patient's heart 16, as previously noted with reference to FIG. 1. Physically, the connections between leads 14 and the various internal components of pulse generator 10 are facilitated by means of a conventional connector block assembly 11, shown in FIG. 1. Electrically, the coupling of the conductors of leads and internal electrical components of pulse generator 10 may be facilitated by means of a lead interface circuit 19 which functions, in a multiplexer-like manner, to selectively and dynamically establish necessary connections between various conductors in leads 14, including, for example, atrial tip and ring electrode conductors ATIP and ARING and ventricular tip and ring electrode conductors VTIP and VRING, and individual electrical components of pulse generator 10, as would be familiar to those of ordinary skill in the art. For the sake of clarity, the specific connections between leads 14 and the various components of pulse generator 10 are not shown in FIG. 3, although it will be clear to those of ordinary skill in the art that, for example, leads 14 will necessarily be coupled, either directly or indirectly, to sense amplifier circuitry 24 and stimulating pulse output circuit 26, in accordance with common practice, such that cardiac electrical signals may be conveyed to sensing circuitry 24, and such that stimulating pulses may be delivered to cardiac tissue, via leads 14. Also not shown in FIG. 3 is the protection circuitry commonly included in implanted devices to protect, for example, the sensing circuitry of the device from high voltage stimulating pulses.

As previously noted, stimulation control circuit 20 includes central processing unit 32 which may be an off-the-shelf programmable microprocessor or micro controller, but in the present invention is a custom integrated circuit. Although specific connections between CPU 32 and other components of stimulation control circuit 20 are not shown in FIG. 3, it will be apparent to those of ordinary skill in the art that CPU 32 functions to control the timed operation of stimulating pulse output circuit 26 and sense amplifier circuit 24 under control of programming stored in RAM/ROM unit 30. It is believed that those of ordinary skill in the art will be familiar with such an operative arrangement.

With continued reference to FIG. 3, crystal oscillator circuit 28, in the presently preferred embodiment a 32,768-Hz crystal controlled oscillator, provides main timing clock signals to stimulation control circuit 20. Again, the lines over which such clocking signals are provided to the various timed components of pulse generator 10 (e.g., microprocessor 32) are omitted from FIG. 3 for the sake of clarity.

It is to be understood that the various components of pulse generator 10 depicted in FIG. 3 are powered by means of a battery (not shown), which is contained within the hermetic enclosure of pacemaker 10, in accordance with common practice in the art. For the sake of clarity in the Figures, the battery and the connections between it and the other components of pulse generator 10 are not shown.

Stimulating pulse output circuit 26, which functions to generate cardiac stimuli under control of signals issued by CPU 32, may be, for example, of the type disclosed in U.S. Pat. No. 4,476,868 to Thompson, entitled “Body Stimulator Output Circuit,” which patent is hereby incorporated by reference herein in its entirety. Again, however, it is believed that those of ordinary skill in the art could select from among many various types of prior art pacing output circuits that would be suitable for the purposes of practicing the present invention.

Sense amplifier circuit 24, which can be of arbitrary conventional design, functions to receive electrical cardiac signals from leads 14 and to process such signals to derive event signals reflecting the occurrence of specific cardiac electrical events, including atrial contractions (P-waves) and ventricular contractions (R-waves). In lieu of such conventional designs a digital signal processor (DSP) sensing amplifier can be utilized. Such amplifiers have several advantages including rapid response time, quick recovery (versus analog) and the like. CPU provides these event-indicating signals to CPU 32 for use in controlling the synchronous stimulating operations of pulse generator 10 in accordance with common practice in the art. In addition, these event-indicating signals may be communicated, via uplink transmission, to external programming unit 20 for visual display to a physician or clinician.

Those of ordinary skill in the art will appreciate that pacemaker 10 may include numerous other components and subsystems, for example, activity sensors and associated circuitry. The presence or absence of such additional components in pacemaker 10, however, is not believed to be pertinent to the present invention, which relates primarily to the implementation and operation of communication subsystem 34 in pacemaker 10, and an associated communication subsystem in external unit 20.

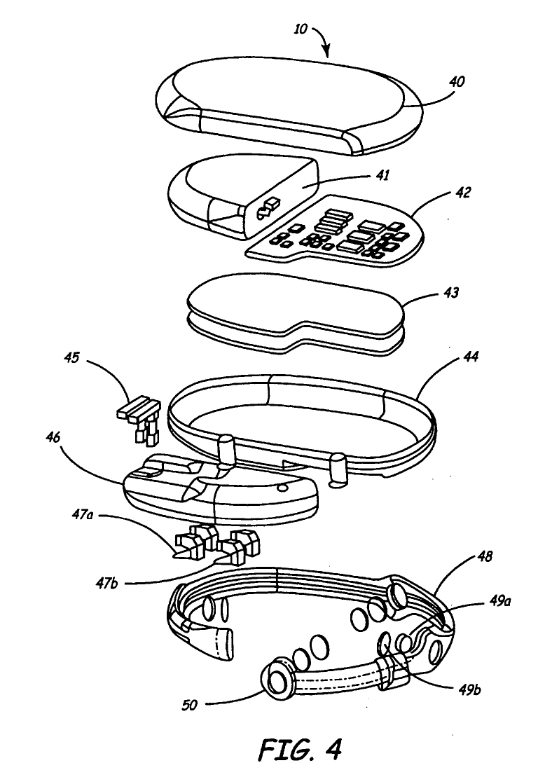

FIG. 4 is a breakaway drawing of a typical implantable cardiac pacemaker 10 in which the present invention is practiced. The outer casing of the pacemaker is composed of right shield 40 and left shield 44. Left shield 44 also has a feedthrough assembly through which wires electrically connecting the lead contacts 47a and 47b to hybrid circuitry 42 are passed. Power to circuitry 42 is provided by battery 41. Pacing leads (not shown) are inserted into lead connector module 46 so that the portion of the lead that leads to the lead ring electrode makes electrical contact with lead contact 47a and lead tip (distal) electrode makes electrical contact with lead contact 47b when lead fastener 46 is turned to its closed position.

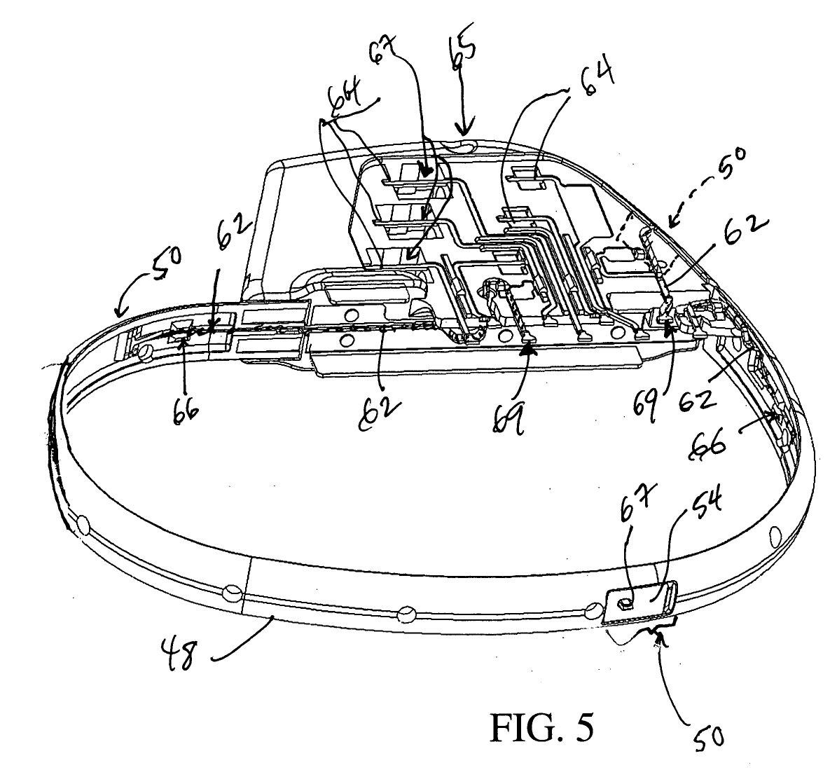

Continuing with FIG. 4, the mechanical portion of the present invention consists of surround shroud 48 that is affixed circumferentially around the perimeter of the implantable pacemaker. In one embodiment of the present invention, there are four recessed openings 50. A cup 49a with a contact plate 49b is fitted into each recessed opening. Into each of recessed openings 50 is placed an electrode such as a flat plate electrode that, in conjunction with other paired electrodes detect cardiac activity. These electrical signals are passed to contact plate 49b that is electrically connected to hybrid circuitry 42 via insulated wires running on the inner portion of surround shroud 48 (see FIG. 5 for details).

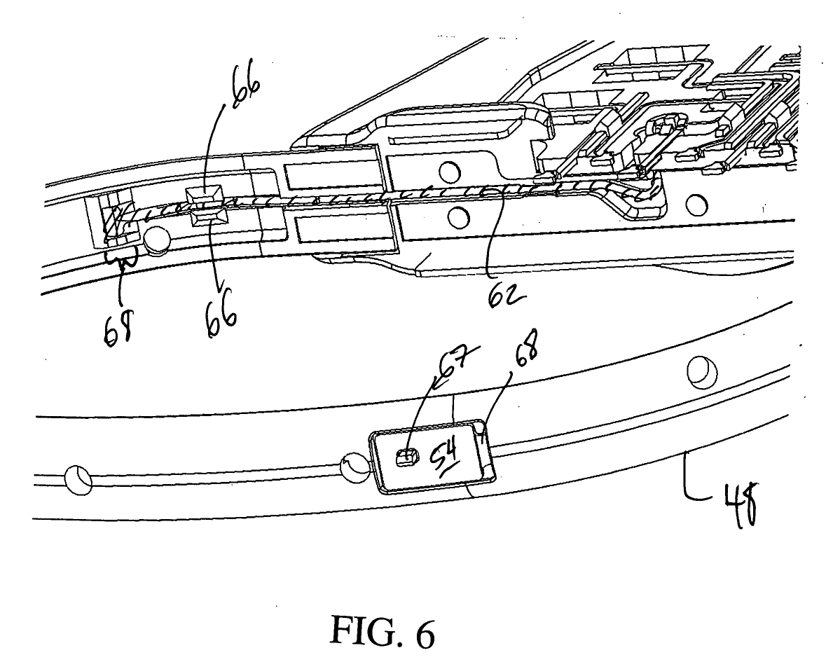

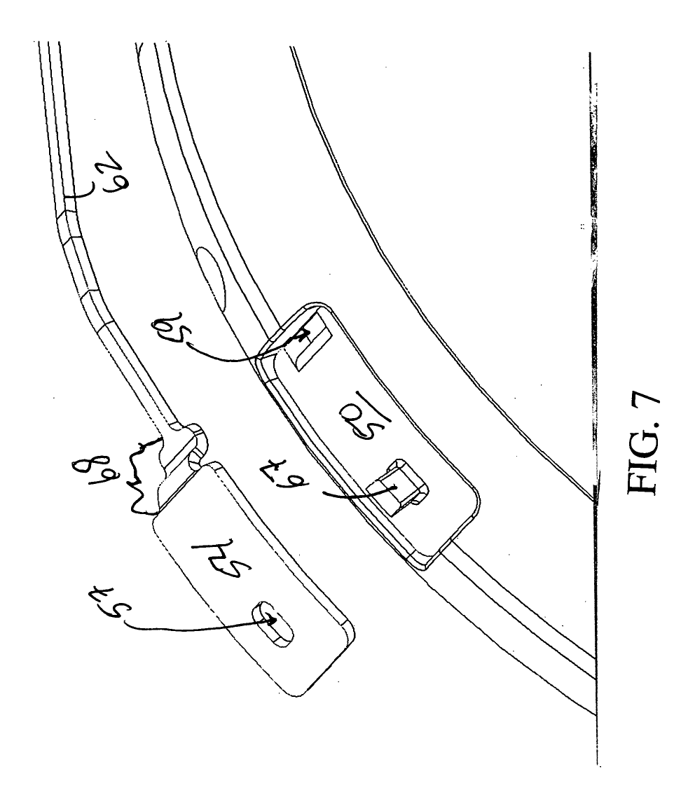

FIG. 5 depicts a perspective view with certain parts removed for ease of reference of a shroud 48 of one embodiment of the invention. In the embodiment depicted in FIG. 5 three substantially planar electrodes 54 couple to corresponding electrode-receiving recesses 50. The recesses optionally include a mechanical fixation feature 67 which can comprise one or more protrusions or bosses oriented to occupy one or more apertures (57 in FIG. 7) formed in the electrode 54. The electrodes 54 establish electrical communication with a distal end portion 69 through elongated conductor 62. In one form of the invention the electrode 54, conductor 62 and distal portion 69 comprise a unitary member that is processed using standard metalworking techniques. In this form of the invention the final shape (for assembly purposes) of the discrete portions of the electrode assembly is attained to thereby speed accurate and rapid final assembly procedures. In another form of the invention one or more of the discrete portions of the assembly comprise individual units that are bonded together to establish electrical communication therethrough. In some embodiments of the invention a transitional portion is included between the electrode 54 and the elongated conductor portion 62 (see FIG. 6-9).

Other embodiments of the invention include one or more members 66 configured to retain a portion of the assembly. Also depicted in FIG. 5 are elongated conductors 64 adapted for coupling to one or more lead-receiving bores 67 formed in a connector module portion 65 of the shroud 48 and/or an IMD which the shroud 48 surrounds.

FIG. 6 depicts another perspective view similar to the view of FIG. 5 except from a slightly different angle and illustrating enlarged components of a shroud 48 of one embodiment of the invention. This view of an embodiment of the invention provides an improved perspective of members 66, protrusion 67, and transitional portion 68 adapted to mechanically retain a portion of the assembly, the plate electrode 54, and a portion adjacent the plate electrode 54, respectively.

FIG. 7 is an exploded view depicting an exemplary electrode 54 adjacent an electrode receiving recess 50 according to one embodiment of the invention. Also depicted in FIG. 7 is an exemplary aperture 57 formed in the electrode 54 for receiving the protrusion 67 as well as the aperture 59 for receiving and, preferably, interlocking with the transitional portion 68. If used in combination the protrusion 67-aperture 57 and the transitional portion 68-aperture 59 provide two discrete fixation locations for the electrode 54. For example the aperture 59 can be located at any portion of the periphery or major part of the recess 50 to provide a discrete retaining force. In addition to or in lieu of the foregoing one of more protrusion members 67 can provide other discrete fixation locations for the electrode 54.

The protrusion 67 can comprise a unitary member adapted to receive an ultrasonic bonding horn to thus form a rivet-like enlarged head portion to increase the fixation of the electrode 54 and/or can comprise a split member which expands after the electrode 54 is fully mounted. Such a split member can include an enlarged head portion for retaining the electrode (with or absent ultrasonic bonding of same).

As known in the art of ultrasonic bonding an ultrasonic head couples to the protrusion 67 which can comprise a thermoplastic or resin-based material and the material quickly deforms; in this case, the material deforms to provide additional mechanical fixation to the substantially planar electrode 54. The operative head of the ultrasonic head can be configured to only impinge upon the protrusion 67 and not with any surrounding part of the shroud 48 (e.g., the edges of the recess 50, etc.). While not specifically depicted herein, in this aspect of the invention the head comprises an effective head portion adapted specifically for producing a weld nugget on the upper portion of protrusion 67. Issued U.S. Pat. No. 6,205,358 entitled “Method of Making Ultrasonically Welded, Staked or Swaged Components in an Implantable Medical Device” and assigned to Medtronic, Inc. describes and depicts some aspects of ultrasonic welding and the entire contents of the '358 patent are hereby incorporated herein. Also, U.S. Pat. No. 6,768,128 entitled “Ultrasonic-Welding Apparatus, Optical Sensor and Rotation Sensor for the Ultrasonic-Welding Apparatus is hereby incorporated herein by reference.

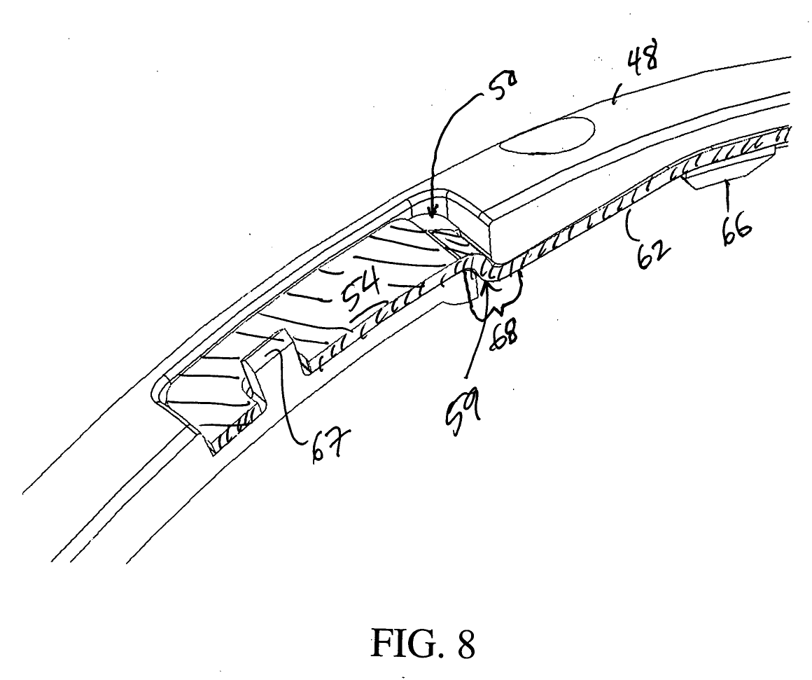

FIG. 8 depicts in perspective view a cross-sectional portion of an electrode-receiving recess 50 having an electrode 54 coupled therein according to one embodiment of the invention. In the depicted embodiment the protrusion 67 comprises an axially split member (with just one half illustrated) as just described. Similarly, only half of the member 66 is depicted due to the cross-sectional view employed. The transitional portion 68 of the electrode assembly is shown effectively interlocked with aperture 59. In the depicted embodiment opposing surfaces of the aperture 59 mechanically cooperate with surface portions of the transitional portion 68 to effectively provide three-dimensional (3D) mechanical support thereto.

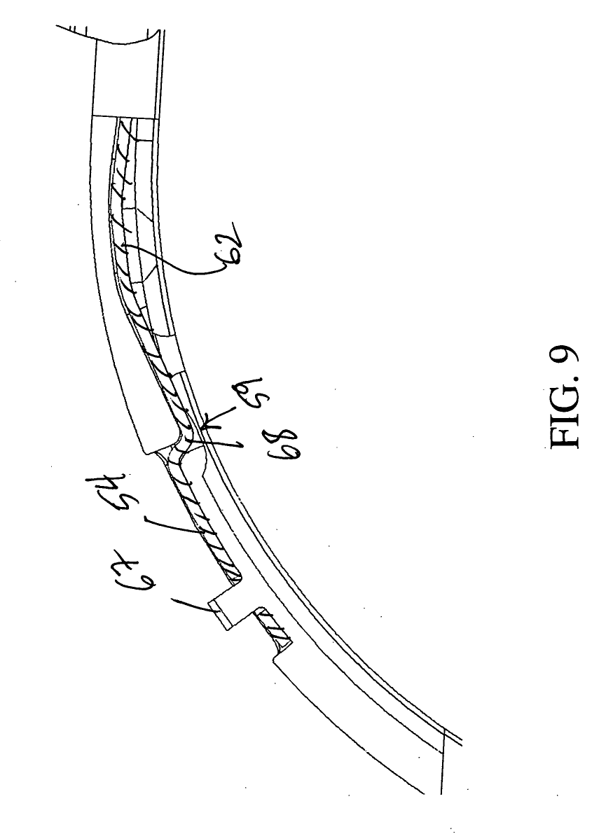

Referring now to FIG. 9, an elevational view in cross-section of the electrode-receiving recess 50 having an electrode 54 coupled therein according to one embodiment of the invention is illustrated. In this view at least two of the 3D mechanical support features of the aperture 59 is depicted. Of course, other shapes and geometries can be effectively utilized to provide such mechanical support, and to the extent that a protrusion member 67 retains the electrode within the recess 50 then the retentions requirements for the interlocking portions 59,68 can be relaxed.

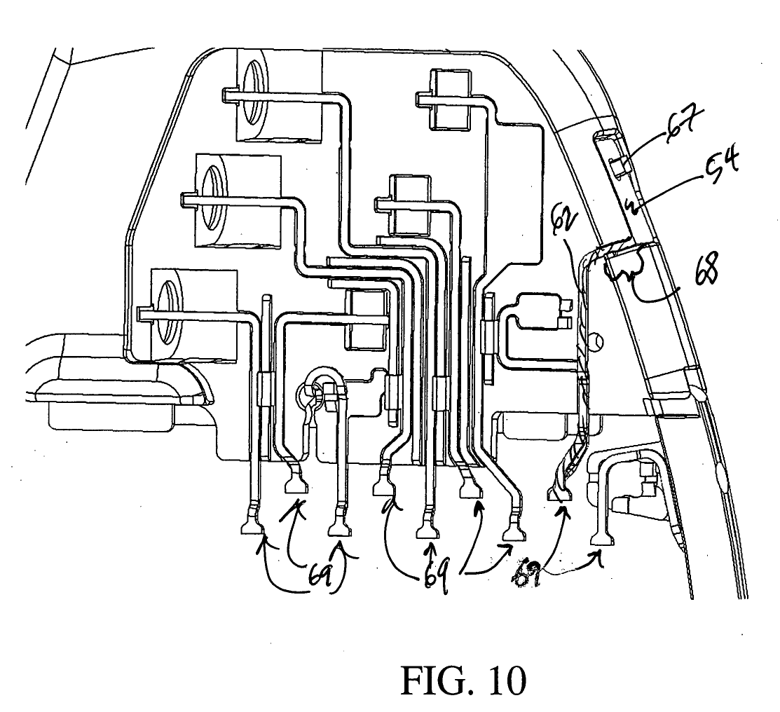

Referring now to FIG. 10, an elevational view of the juxtaposition of a plurality of distal end portions 69 of the elongated conductors 62 of the electrodes 54 following assembly of a shroud 48 assembly according to one embodiment of the invention is presented. In this view an electrode 54 couples via protrusion 67 and a peripheral, side-oriented transitional portion 68 (relative to electrode 54) transitions to elongated conductor portion 62. Portion 62 terminates at distal tip portion 69 in a predetermined relationship to a plurality of other distal tip portions (also labeled 69 for convenience) including some portions emanating from other than shroud-based electrodes 54. According to this aspect of the invention the plurality of tip portions 69 are commonly and accurately presented for connection to corresponding upwardly extending conductive pins typically extending through the IMD housing (e.g., a multi-polar feedthrough array) which are utilized in conjunction with capacitive filters to maintain hermeticity of the IMD while providing electrical communication with external components.

Of course, the electrodes can be fabricated out of any appropriate material, including without limitation tantalum, tantalum alloy, titanium, titanium alloy, platinum, platinum alloy, or any of the tantalum, titanium or platinum group of metals whose surface may be treated by sputtering, platinization, ion milling, sintering, etching, or a combination of these processes to create a large specific surface area. Also as noted herein, an electrode can be stamped, drawn, laser cut or machined using electronic discharge apparatus. Some of the foregoing might require de-burring of the periphery of the electrode or alternately any sharp edges due to a burr can be coupled facing toward the corresponding recess in the shroud member thereby minimizing likelihood of any patient discomfort post-implant while further reducing complexity in the fabrication of assemblies according to the invention. The electrodes can be coated or covered with platinum, a platinum-iridium alloy (e.g., 90:10), platinum black, titanium nitride or the like.

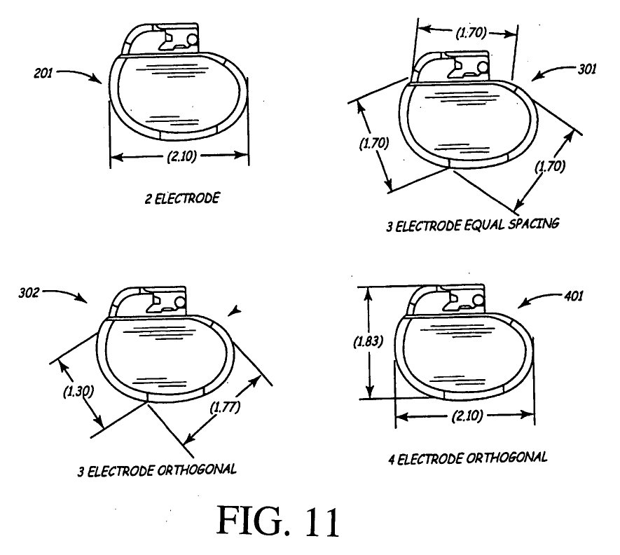

FIG. 11 is an illustration of the various possible electrode sites that may be located along the perimeter of the implanted pacemaker within the compliant shroud. The spacing of the electrodes display the measurements depicted in Table 1. The spacings, as shown, also illustrate the vectors that may be used to detect the cardiac depolarizations. For example, the orthogonal 3-electrode design 302 requires only two potential vectors, as opposed to the equal spacing 3-electrode design 301 that may require the use of all three vectors. A more detailed analysis of these geometries may be found in U.S. Pat. No. 6,505,067 to Lee et al. entitled, “System and Method for Deriving a Virtual ECG or EGM Signal,” the contents of which are fully incorporated herein.

Accordingly, a number of embodiments and aspects of the invention have been described and depicted although the inventors consider the foregoing as illustrative and not limiting as to the full reach of the invention. That is, the inventors hereby claim all the expressly disclosed and described aspects of the invention as well as those slight variations and insubstantial changes as will occur to those of skill in the art to which the invention is directed. The following claims define the core of the invention and the inventors consider said claims and all equivalents of said claims and limitations thereof to reside squarely within their invention.

1. A physiologic signal acquisition system including at least one subcutaneous electrode coupled to operative circuitry disposed within a hermetically sealed housing, comprising:
a hermetically sealed housing for an implantable medical device (IMD);
a shroud member adjacent at least a part of the peripheral portion of the housing, said shroud member including at least one recessed region adapted to receive a substantially planar, plate-type electrode, wherein the substantially planar, plate-type electrode mechanically interlocks to one of: a peripheral feature and a non-peripheral feature of the at least one recessed region; and
signal processing circuitry mounted inside the housing and electrically coupled to the electrode to detect the cardiac signals.

2. A system according to claim 1, wherein the peripheral feature or the non-peripheral feature of the at least one recessed region comprises at least one of: an aperture, a protrusion.

3. A system according to claim 2, wherein the protrusion comprises one of a unitary member and an axially-bifurcated member.

4. A system according to 3, wherein the bifurcated member includes an enlarged head portion.

5. A system according to 3, wherein the protrusion is formed from a thermoplastic material that is susceptible of one of: ultrasonic welding and deforming in response to applied ultrasonic energy.

6. A system according to claim 2, wherein the aperture comprises a serpentine bore adapted to receive an elongated member and said elongated member couples to a portion of the electrode.

7. A system according to claim 6, wherein a portion of the elongated member is adapted to mechanically interlock with at least a corresponding portion of the serpentine bore.

8. A system according to claim 6, further comprising a mechanical retention member adapted to mechanically interlock with at least a corresponding portion of the elongated member.

9. A system according to claim 6, wherein the mechanical retention member comprises a pair of opposing, resilient spaced-apart members.

10. A system according to claim 1, further comprising a biocompatible coating disposed over at least an exposed surface of the substantially planar, plate-type electrode.

11. A system according to claim 10, wherein the coating comprises one of:
a nitride coating, a platinum coating, a coating of platinum black, a drug-eluting coating, a steroidal coating.

12. A system according to claim 1, wherein the IMD comprises one of:
an implantable pacemaker including at least one medical electrical lead adapted for endocardial deployment, an implantable cardioverter-defibrillator (ICD), a drug delivery pump, a subcutaneous ICD, a submuscular ICD, a brain stimulation device, a nerve stimulation device, a muscle stimulation device.

13. A system according to claim 1, further comprising:
a plurality of cardiac depolarization sensing electrodes disposed into the housing and coupled to the electrode;
means for storing signals derived between the electrode and at least two other electrodes; and
means for determining a highest-magnitude sensing vector from among the stored signals.

14. A system according to claim 13, wherein the at least two other electrodes are also coupled to the shroud member in a spaced-apart relation.

15. A system according to claim 14, wherein said electrode and said at least two other electrodes are spaced-apart to provide maximal electrode sensing.

16. A system according to claim 8, wherein said electrode spacing includes a spacing of three vectors with a separation angle of approximately 120° therebetween to thereby form triangular spacing between electrode pairs for signal variation minimization.

17. A system according to claim 8, wherein said electrode spacing includes electrodes arranged around the shroud member thus forming an equilateral triangle along the perimeter of the IMD.

18. A system according to claim 1, wherein the substantially planar, plate-type electrode comprises a member formed of at least one of:
a tantalum material, a titanium material, a platinum material.

19. A system according to claim 6, further comprising a potting material disposed in contact with at least a portion of the elongated member and wherein the potting material comprises one of a medical adhesive and a biocompatible dielectric material.

20. A system for monitoring cardiac activity with electrodes spaced from myocardial tissue, comprising:
an electrode-receiving recess formed in the periphery of a non-conductive biocompatible shroud member; and
a substantially flat electrode having opposing major surfaces disposed within a major lower portion of the recess and mechanically interlocking with at least two parts of said recess, a first aperture-part and a second protrusion-part.
